(19) United States
(12) Patent Application Publication
(10) Pub. No.: US 2026/0087912 A1
(43) Pub. Date: Mar. 26, 2026

(54) Haptic Feedback Device

(71) Applicant: Nottingham Trent University, Nottingham, Nottinghamshire (GB)

(72) Inventors: Don Pasindu Vijai Lugoda, Nottingham, Nottinghamshire (GB); Richard Charles Arm, Nottingham, Nottinghamshire (GB)

(73) Assignee: Nottingham Trent University, Nottingham, Nottinghamshire (GB)

(21) Appl. No.: 19/109,953

(22) PCT Filed: Sep. 4, 2023
(86) PCT No.: PCT/GB2023/052277

§ 371 Date: Mar. 7, 2025

(30) Foreign Application Priority Data
Sep. 7, 2022 (GB) 2213072.8

(51) Int. Cl. G08B 6/00

(52) U.S. Cl. CPC G08B 6/00

(57) ABSTRACT

This invention provides for a haptic feedback device comprising a body having a body cavity surrounded by a layer of non-expandable material having an interior and exterior surface and which comprises at least one aperture therethrough, and an expandable material located on the exterior surface or interior surface of the non-expandable material and covering at least one aperture, and wherein the device further comprises at least one connector for connecting the device, in use, to a source of fluid.

### TECHNICAL FIELD OF THE INVENTION

The present invention relates to a haptic feedback device, a haptic feedback system, a textile article comprising a haptic feedback device and a process for creating a haptic feedback response.

### BACKGROUND TO THE INVENTION

Haptic feedback devices simulate the sense of touch. Creating realistic haptic feedback using technology can not only create a realistic experience in a virtual scenario such as gaming, it can also be used to virtually reproduce a scenario to help with training for many different applications, including medicine, aviation, robotics, electronic displays and the motor industry. Haptic feedback devices often utilise vibrotactile or electrotactile technologies which can be cheap and easily powered; however, there are often difficulties in realistically simulating forces felt, for example when gripping an item, when using many known haptic devices.

Haptic feedback can be created using a fluidic system such as a pneumatic or a hydraulic system (amongst others). Examples of pneumatic haptic feedback systems are disclosed in U.S. Pat. No. 10,233,910B2 and WO2017120314A1. These haptic systems comprise a bladder within a flexible layer which results in the flexible layer bending once the bladder is inflated. These disclosures have the disadvantage of not being able to recreate a sensitive or location specific haptic response. This in turn can limit the realistic haptic feedback experienced by the user. It would therefore be advantageous to create a haptic feedback device that can create force feedback responses in precise, localised positions which may be representative of real-life scenarios created in a virtual environment.

It may also be advantageous to create a simple haptic feedback device that can be easily manufactured and integrated into items such as clothing which can create a realistic haptic feedback response.

It may also be advantageous to create a localised haptic feedback system made from durable materials such that it can withstand mechanical rigor.

It may also be advantageous to create a localised haptic feedback system that can be integrated into an item such as an item of clothing or can be retroactively fitted onto the item.

It is an aim of embodiments of the invention to overcome one or more problems of the prior art, whether expressly disclosed herein or not.

### SUMMARY OF THE INVENTION

According to a first aspect of the invention, there is provided a haptic feedback device comprising a body having a body cavity surrounded by a layer of non-expandable material having an interior and exterior surface and which comprises at least one aperture therethrough, and an expandable material located on the exterior surface or the interior surface of the non-expandable material over at least one aperture, and wherein the device further comprises at least one connector for connecting the device, in use, to a source of fluid.

The haptic feedback device according to the invention may be advantageous because it can create a haptic feedback response for the user such that the user may feel like they are touching an object or holding an item or in contact with something or someone in a virtual reality scenario. The haptic feedback device according to the first aspect of the invention may be advantageous because it may be used with a source of fluid wherein the fluid is a gas or a liquid.

The expandable material is defined as a material that can stretch and expand. The non-expandable material is defined as a material with limited stretchability which cannot expand.

In preferred embodiments the expandable material is located on the exterior surface of the non-expandable material. In some embodiments, the non-expandable material is in the form of a layer, a sheet or a film. In some embodiments the non-expandable material forms a layer substantially enclosing the hollow cavity. In some embodiments the non-expandable material is a flexible non-expandable material. The flexible non-expandable material can bend, drape and sheer but it has limited stretchability so it cannot expand. The use of a flexible, non-expandable material may be advantageous because it may bend easily without breaking such that it may fit around a user's body part or area of skin for which a haptic response is required to be felt, in use.

In some embodiments the non-expandable material may be a non-expandable textile material. In some embodiments the non-expandable textile material may be a woven fabric, a knitted fabric or a non-woven fabric, such as a non-woven felted fabric. In some embodiments the non-expandable textile may comprise at least one material selected from the group consisting of polyester, nylon, cotton, Kevlar, cordura, wool, carbon fibre, glass fibre, silk or any other textile with limited stretchability, or combinations thereof. A non-expandable textile may be advantageous because it may be flexible such that it can easily be formed into any desired shape of haptic feedback device body and when used in any application, it can be formed into suitable shapes for use and flexibly bent or moved as required.

An aperture may also be described as a gap or hole surrounded by an edge or rim. In some embodiments the aperture is surrounded by an unbroken edge or rim comprising the non-expandable material. The apertures may be any suitable shape Examples of suitable shapes include but are not limited to a circle, an oval, a quadrilateral or a triangle. The size and shape of the aperture may reflect the application of the haptic feedback device. Haptic feedback devices for certain applications may comprise a large number of small apertures. This may be advantageous because it may create a precisely localised, sensitive haptic feedback response. Haptic feedback devices for other applications may comprise larger apertures. This may be advantageous to create a haptic feedback response such as simulating an interaction with a virtual environment for example sitting, leaning or resting on a surface or holding a large item.

There may be individual portions of expandable material covering all apertures, or there may be a single layer of expandable material covering all apertures. In some embodiments, the expandable material is in the form of a layer which covers substantially the whole of the exterior surface of the non-expandable material. In some embodiments the expandable material is in the form of a layer which covers at least part or substantially the whole of the interior surface of the non-expandable material. In some embodiments the expandable material is in the form of a layer which covers at least part or substantially the whole of the exterior surface of the non-expandable material. The expandable material may comprise an interior and an exterior surface wherein the interior surface of the expandable material is adjacent to and contacts the exterior surface of the non-expandable material. The exterior surface of the expandable material may be adjacent to and in contact with the interior surface of the non-expandable material.

In some embodiments the expandable material may be attached or connected to the non-expandable material. In some embodiments the expandable material and non-expandable material may be located next to each other but not attached or connected. In some embodiments a portion of the expandable material may be integrally connected to the non-expandable material, for example it may be adhered, embedded, encapsulated, melded or entrained within a portion of the non-expandable material. In embodiments wherein the non-expandable material is a porous material, the expandable material may be located next to the non-expandable material and the expandable material may be embedded or encapsulated within the pores of the non-expandable material. Since the expandable material above or below the or each aperture is not connected to the non-expandable material at that position, the expandable material is free to expand or inflate when fluid pressure is exerted through the aperture and against the expandable material, which then forms a protrusion or bubble of expandable material which can provide haptic feedback to an area of a user's skin or body, which in use is adjacent to the bubble or protrusion.

In some embodiments the expandable material may be a natural or synthetic elastomer. In some embodiments the expandable material may be a thermally cured or a cross-linked polymer. In some embodiments the expandable material may comprise at least one elastomer selected from the group consisting of fluorosilicone, polytetrafluoroethylene, polydimethylsiloxanes (PDMS), polyethylene, polyvinyl, polyurethane, styrene butadiene, nitrile, butyl rubber, natural latex rubber, ethanol vinyl acetate, polyvinyl chloride, or any combination thereof. Elastomers may be advantageous because they are able to stretch by variable amounts depending on the pressure exerted on the material thereby creating a haptic feedback response of variable magnitude. Elastomers may also have good recovery properties such that they return to their starting shape quickly and easily and can therefore be used repeatedly for a long period of time before replacement is required. This may result in a longer lasting product which in turn may reduce waste and inconvenience to the user.

In some embodiments the body of the haptic feedback device may comprise a plate located between the non-expandable and expandable materials and over at least two apertures within the non-expandable material. The plate may be a non-expandable, flexible plate. The plate may be a non-expandable textile material. The plate may be the same material as the non-expandable material. The plate may alternatively be polyethylene terephthalate or an imide such as Kapton polyimide. This embodiment may be advantageous because it may create a haptic feedback response with a larger surface area whilst also maintaining flexibility. This embodiment may be advantageous for creating a haptic feedback response for example in the palm of a user's hand or any large surface such as the abdomen, back, arms or legs

In some embodiments a second, expandable layer may be located above the exterior surface of the expandable material over the or each aperture of the haptic feedback device body. The second expandable layer may be located only above the expandable material of the haptic feedback device body, or may in some embodiments cover substantially the entire haptic feedback device body. The second expandable layer may be less expandable than the expandable material of the haptic feedback device body. The second expandable layer may be an expandable textile which may be defined as any fabric which can expand to a greater length or width than the starting length or width of the fabric and can return to the original shape. The second expandable layer may comprise elastane, spandex or Lycra®. The second expandable layer may have less stretch than the expandable material of the body such that it restricts the expansion or stretch of the expandable material of the body however it may still have limited stretch such that when the haptic feedback device protrusions are inflated, the second expandable layer may also move. This embodiment may be advantageous because the second expandable material may act as an extensibility limiter, and therefore prevent the expandable material of the body, located at the cavity of the haptic feedback device, from expanding beyond its elastic limit which may negatively result in the expandable material not returning to its original configuration after cessation of pressure, or rupturing. The second expandable material may also be advantageous as a protection layer against environmental or user damage. The presence of the second expandable material may therefore be advantageous in extending the lifetime of the haptic feedback device. This embodiment may be advantageous because the second expandable material layer may move a limited amount as the expandable material located at the cavity of the haptic feedback device inflates thereby protecting the haptic feedback device without hampering the haptic feedback response in use.

In other embodiments there may be a second non-expandable layer located above the exterior surface of the expandable material over the or each aperture of the haptic feedback body, wherein the second non-expandable layer may be configured to enable expansion of the expandable material to a limited extent when pressure is forced through the aperture over which the expandable material is located. Such a layer may comprise a layer of non-expandable material which is connected around the expandable material over the or each aperture but not connected to the expandable material over the or each aperture and sized to enable expansion of the expandable material to a defined or limited degree when pressure is exerted through the or each aperture.

In some embodiments the body of the haptic feedback device may comprise a hollow tube formed by the non-expandable material around the cavity of the body. In some embodiments the tube may comprise a textile yarn or fibre. In some embodiments the tube may comprise a circular cross-section. In some embodiments the tube may comprise an oval cross-section. In some embodiments the tube may comprise a square or any other shape cross-section. The non-expandable material may be in the form of a layer, a sheet or a film. The layer, sheet or film of the non-expandable material may be surrounded by the expandable material. The expandable material may be in the form of a layer, a sheet or a film. The expandable material may be located above the apertures in the non-expandable material and may completely surround the non-expandable material to form a layer of the tube around the non-expandable material of the body. A tube may be advantageous because it can be integrated into clothing, soft furnishing or any wearable items, such as straps or pads, such that a haptic feedback response can be created when the wearable item or soft furnishing is in contact with a user.

In some embodiments the body of the haptic feedback device may comprise a sheet comprising a top side and a bottom side connected around outer edges thereof, wherein the cavity of the body is located between the top side and bottom side. The top side may comprise the non-expandable material of the body comprising apertures. The bottom side may comprise the non-expandable material of the body comprising apertures. In some embodiments both the top and bottom sides comprise the non-expandable material of the body wherein at least one layer of the non-expandable material comprises at least one aperture. Each of the top and bottom sides comprises an exterior and interior surface, with the interior surface facing the cavity of the body. The expandable material may be located on the exterior or interior surface of the top and/or bottom side but is preferably located on the exterior surface. When the expandable material comprises a layer of expandable material, this may form a sheet or layer on the exterior surface of the top and/or bottom side of the non-expandable material and is located above or below the apertures. A haptic feedback device comprising a body in the form of a sheet may be advantageous because it enables a haptic feedback response over a large surface area.

In some embodiments the fluid connector of the haptic feedback device may comprise a fluid inlet connector or a fluid outlet connector. An inlet connector may allow fluid to move into the cavity of the haptic feedback device. An outlet connector may allow fluid to move out of the cavity of the haptic feedback device. The fluid may be a gas or a liquid. The inlet connector and outlet connector may comprise connectors for connecting gaseous or liquid fluid, so as air or water, for example. A suitable connector may be a tube-to-tube adaptor, a barbed straight connector pipe hose, joiner tubing, 1,2 or 3-way mini electric solenoid valve, a pneumatic PCL type connector or a push in pneumatic connector or any other suitable mechanical or one way valve. The inlet connector and outlet connector may be located spaced apart from each other, for example at opposite sides of the body of the device.

According to a second aspect of the invention there is provided a haptic feedback system comprising a haptic feedback device according to the first aspect of the invention, connected to a fluid source.

In some embodiments the fluid source may be a source of a pressurised gas such as pressurised air, pressurised carbon dioxide, pressurised nitrogen or any other suitable pressurised gas. The source of pressurised gas may be a central pipeline, a tank or a cannister. In some embodiments the source of pressurised air may be a compressed air canister or a carbon dioxide cannister.

In some embodiments the system may comprise a fluid pump connected between the haptic feedback device and the fluid source. When the fluid source is air, the pump may comprise an air pump. In other embodiments, when the fluid source is an aqueous fluid, the pump may comprise a hydraulic pump.

The fluid source may comprise an outlet. In some embodiments the fluid source outlet may be connected to at least one pipe which in turn may be connected to the fluid inlet located at or on the body of the haptic feedback device such that fluid can be transferred from the fluid source to the body cavity in the body of the haptic feedback device.

The fluid source may comprise an inlet. In some embodiments the fluid source inlet may be connected to at least one pipe which in turn may be connected to the fluid outlet located at or on the body of the haptic feedback device such that fluid can be transferred back to the fluid source from the body cavity in the haptic feedback device.

In some embodiments the fluid source may be a removable part of the haptic feedback system. This may be advantageous should the fluid source need to be changed from one gas to another, one liquid to another or from liquid to gas or vice versa. This may also be advantageous for large scale fluid sources as it allows the device to be disconnected from the fluid source after use or between uses and should the fluid source be a cannister or fluid container the container may be replaced and/or refilled once it has been emptied.

The haptic feedback system may comprise at least one valve located at any point in the haptic feedback system, which in use enables control of the flow of fluid from and to the fluid source. In some embodiments the haptic feedback system may comprise at least one mechanical valve. In some embodiments the haptic feedback system may comprise at least one electronically controlled valve wherein the electronically controlled valve is actuated by electronic signals sent by an electronic control unit. In some embodiments the haptic feedback system may comprise at least one mechanical valve and at least one electronically controlled valve.

In some embodiments the valve may be a one-way valve, a two-way valve, or a three-way valve. In some embodiments the valve may be a one-way solenoid valve. A one-way valve may be advantageous because it may allow priming wherein the fluid may to be transferred from the fluid source into the body cavity without backflow thereby allowing pressure to be maintained in the body cavity between actuation periods. A one-way valve may be advantageous if there is a large distance from the fluid source to the body of the haptic feedback device as it would allow for the pressure and direction of the fluid to be controlled and maintained between actuation periods. An electronically controlled valve may be advantageous because it would allow the flow of fluid from the fluid source to the haptic feedback device to be controlled externally. More than one valve may be advantageous if there is a large distance from the fluid source to the haptic feedback device because it may allow accurate fluid pressure control thereby resulting in a more accurate haptic feedback response.

In some embodiments the electronically controlled valve may be a solenoid valve. In some embodiments the solenoid valve may be an electronically controlled inlet slave solenoid valve. A solenoid valve may be advantageous because it may control the flow of the fluid such that enough fluid enters the haptic feedback device to create a haptic feedback response. A solenoid valve may also be advantageous because it may prevent too much fluid from entering the haptic feedback device therefore preventing damage to the device. An electronically controlled valve may be advantageous because it may be controlled externally by an electronic control unit.

In some embodiments, in which the fluid source comprises a fluid source outlet, the fluid source outlet may comprise an adjustable valve to control the flow of the fluid out of the fluid source. In some embodiments the adjustable valve may be an adjustable flow rate screw valve. The use of an adjustable valve enables control of the flow rate of the fluid out of the fluid source to the haptic feedback device and vice versa such that required pressure can be maintained and varied depending on use parameters.

In some embodiments wherein the fluid connector comprises a fluid outlet, the fluid outlet may be connected to an outlet valve. In some embodiments the outlet valve may be actuated by electronic signals. In some embodiments the fluid outlet may be connected to an outlet pipe which in turn may be connected to an outlet valve. In some embodiments the fluid outlet valve may be an exhaust valve or an exhaust solenoid valve to vent the fluid out of the haptic feedback system. This may be advantageous because the fluid may be removed after the haptic feedback response has been created to bring the haptic feedback body to a resting state between haptic feedback events.

In some embodiments the haptic feedback system may comprise an electronic control unit. In some embodiments the electronic control unit may be a microcontroller or a control module. In some embodiments the microcontroller may be an Arduino, raspberry pi, teensy or any other microcontroller. The electronic control unit may be connected to at least one valve located at any point in the haptic feedback system. The electronic control unit may be connected to at least one valve located between the fluid source and the haptic feedback body such that the fluid flow from the fluid source to the haptic feedback device body can be electronically controlled. The electronic control unit may be connected to at least one valve located at or downstream of the fluid outlet of the fluid connector of the haptic feedback body such that the fluid flow from the haptic feedback device body can be electronically controlled. The electronic control unit may be connected to an outlet valve such as an exhaust valve such that the fluid flow out of the haptic feedback system can be controlled. This may be advantageous because it may allow the flow of the fluid, and therefore the internal pressure of the haptic feedback device, to be controlled externally.

In some embodiments the electronic control unit may be connected to a computer or a virtual reality device or both. In some embodiments the electronic control unit may be connected to the computer or virtual reality device by wires or wirelessly. In some embodiments the computer may transmit and receive electronic signals to the electronic control unit to inflate or deflate the haptic feedback device. The haptic feedback device connected to a computer may be advantageous because it would allow the haptic feedback responses to be coordinated to 2D computer or 3D virtual reality events for uses such as gaming, entertainment, training exercises, education, simulators, communication, physiotherapy, monitoring or any other use wherein the user's sense of touch may be engaged. This embodiment may also be advantageous for secondary and tertiary users such as communication receivers wherein haptic feedback responses can be received and responded to.

In some embodiments the haptic feedback system may comprise at least two haptic feedback devices each connected to one fluid source such that fluid may flow from one fluid source to a plurality of haptic feedback devices. In some embodiments the haptic feedback system may comprise at least two haptic feedback devices each connected to at least one fluid source such that fluid may flow from the same or different fluid sources to a plurality of haptic feedback devices. In some embodiments the haptic feedback system may comprise at least two haptic feedback devices each connected to each other by way of a connector and optionally at least one valve. The connectors may be connected to each other at locations between the haptic feedback devices and the fluid source. The connectors may be connected by one or more valves such as two-way valves or three-way valves. In some embodiments the two or more haptic feedback devices may be arranged in parallel, perpendicularly, end-to-end, superposed or in any other 3D arrangement. A haptic feedback system comprising a plurality of haptic feedback devices may be advantageous because a haptic feedback response may be created over a larger surface area and/or over different areas of skin or different body parts of a user. It may also be advantageous because it may allow for a large number of haptic feedback responses to occur in parallel and independently from other haptic feedback responses in the system through the independent control of valves.

According to a third aspect of the invention there is provided a textile article comprising at least one haptic feedback device according to the first aspect of the invention.

In some embodiments the haptic feedback device may be the haptic feedback device of a haptic feedback system according to the second aspect of the invention.

In some embodiments the textile article may be a wearable textile item. In some embodiments the wearable textile item may be an item of clothing. In some embodiments the wearable textile item may be a glove, a sleeve, a sock, trousers, a hat, a shirt, underwear or a vest. In some embodiments the textile article may be a textile strap that can be attached to the body of a person at any point such as the wrist, the chest, the abdomen, the back, the arms, the legs, the hands or the feet.

In some embodiments the textile article may be part of a soft furnishing item. In some embodiments the soft furnishing may be located on a chair, a bed, a cushion or any other item which may be in contact with a user or may be a soft furnishing item such as a blanket. This may be advantageous because it may create an item wherein when the user is in contact with the item, they can experience a haptic feedback response.

In some embodiments the textile article may comprise a channel or a pocket wherein the haptic feedback device may fit. The channel or pouch may be larger than the haptic feedback device such that the haptic feedback device in its deflated form is entirely within the channel or pocket, or does not protrude from the channel or pocket, but when inflated the textile article may move with the formed protrusions such that in use, the haptic feedback response is felt by the user's skin or body which is in contact with the textile article. The channel or pocket may be located on the inside or the outside of the textile article. The channel or pocket may be retroactively fitted onto or into the textile article such that the haptic feedback device may be added to the textile article after the textile article has been manufactured. This embodiment may also be advantageous because the haptic feedback device may be removable should it require replacing.

In embodiments wherein the haptic device is a tube or yarn, the haptic feedback device may be integrated into a textile article by a method such as knitting, weaving or braiding. This embodiment may be advantageous because the haptic device may be accurately located within the textile article such that accurate and precise haptic feedback responses may be created at any desired part of the textile article wherein the haptic feedback device is integrated.

In some embodiments the haptic feedback device may be integrated with or fitted onto the textile article and the device may be connected by at least one pipe, and optionally at least one valve, to a fluid source. In some embodiments the pipe, valve and fluid source may be attached to the device in the textile article by way of a channel, pouch or pocket formed on or connected to the textile article, and which may comprise the same or different material to the material of the textile article. Such embodiments may be advantageous because the location of the textile article may not be restricted by any pipes or tubes that may connect the haptic feedback device to the fluid source. These embodiments may also be advantageous for a wearable textile article because the user's movements may not be restricted by the connection of the haptic feedback device to a static or large fluid source.

In some embodiments a haptic feedback system integrated with or fitted onto the textile article may be connected to an electronic control unit. In some embodiments the electronic control unit may be a small or micro electronic control unit and may be attached to the textile article such that it wirelessly communicates with a computer or a virtual reality device.

In some embodiments the textile article may comprise multiple haptic feedback devices.

In some embodiments the textile article comprises a haptic feedback device comprising at least one aperture wherein the size and shape of the aperture reflects the anatomical region of interest, and the contact area. By way of example, in embodiments wherein the textile article is a glove with fingers and a palm, the apertures may be circular or oval shaped over the finger segments and the apertures that cover the palm may be square, rectangular, rhomboid, trapezium or triangular. In embodiments wherein the textile article is a t-shirt, vest or other larger item of clothing, the haptic feedback device may comprise a large number of different shaped and sized apertures such that a precise haptic feedback response can be created in the surface covered by the textile article.

In some embodiments the haptic feedback system of the second aspect of the invention may comprise a textile article of the third aspect of the invention.

In some embodiments of the textile article, the haptic feedback device or devices may be connected by way of a haptic feedback system according to the second aspect of the invention to an electronic control unit which may be connected to a computer. The computer and electronic control unit may be arranged to transmit and receive electronic signals to independently control at least one valve within the haptic feedback system such that the haptic feedback device or devices may be independently inflated and deflated. This may be advantageous because it may result in a textile article comprising a haptic feedback device, which may be used for gaming, training or other virtual scenarios, wherein the computer can control the desired haptic feedback to create a realistic environment. Examples of a textile article might be a glove, a sleeve, a shirt or the surface of a chair which can create a haptic feedback response for the user such that the user may feel like they are touching an object or holding an item or in contact with something or someone in a virtual reality scenario.

According to a fourth aspect of the invention there is provided a process for creating a haptic response using a haptic feedback device according the first aspect of the invention comprising the steps of a) connecting the device to a fluid source; and b) inflating the expandable material over at least one aperture with fluid from the fluid source.

The haptic feedback device may be part of a haptic feedback device system according to the second aspect of the invention.

The haptic feedback device may be attached to a textile article according to the third aspect of the invention.
Step a) may comprise connecting the inlet connector of the haptic feedback device by way of at least one pipe to the fluid source such that fluid can be transferred from the fluid source to the haptic feedback device. The pipe may comprise at least one valve according to the second aspect of the invention.

In some embodiments there may be a step after step a) and before step b) comprising opening a fluid source outlet such that fluid may be transferred from the fluid source to the haptic feedback device. In some embodiments there may be a step after step a) and before step b) comprising opening a valve or valves located between the fluid source outlet and a haptic feedback device fluid inlet connector. The valve or valves may be electronically controlled; therefore, there may be a step before step a) or between step a) and step b) wherein the haptic feedback system is connected to an electronic control unit which may in turn be connected by means of wires or wirelessly, to a computer and optionally a virtual reality device. The valve or valves may be mechanical in which case in a step before step a) or between step a) and step b) the mechanical valves may be mechanically opened. There may be more than one valve wherein at least one valve is electronically controlled and at least one valve is mechanically controlled.
Step b) may comprise the fluid entering the haptic feedback device through the haptic feedback device fluid inlet connector such that the body of the haptic feedback device is filled with the fluid. In some embodiments the fluid may be a gas or a liquid. In some embodiments the fluid may enter the aperture(s) in the non-expandable material and come into contact with the expandable material located above the apertures such that the expandable material over at least one aperture may inflate and create a protrusion. The protrusion creates a haptic feedback response.

In some embodiments the haptic feedback device may comprise more than one aperture wherein each aperture is covered by the expandable material such that a protrusion may form above at least one, some or all of the apertures. In embodiments where the expandable material is located below the aperture, the expandable material may be inflated such that it forms a protrusion through and above the aperture.

The fluid flow may be controlled by at least one valve in the haptic feedback system such that the size of the protrusions, the duration of inflation and timing of one, some or all of the protrusions being created may be controlled. The inflation of the or each protrusion may be controlled such that they are independently inflated. Such embodiments may be advantageous because a haptic feedback response can be created which is designed to mimic a simulation or a scenario. This may be useful to create an immersive experience for experiences such as gaming, training or virtual scenario demonstrations.

The haptic feedback system arranged in use to create a haptic response may be suitable for a number of applications including but not limited to gaming, entertainment, training exercises, simulators, communication such as communication with visually impaired or deaf blind people, physiotherapy for example through the use of mouldable orthopaedic casts, surgical robotic training applications or interactive/point of view video, movies and role-play for training and entertainment purposes.

According to a fifth aspect of the invention there is provided a process for creating a haptic response using a haptic feedback system according the second aspect of the invention comprising the step of inflating the expandable material over at least one aperture with fluid from the fluid source.

According to a sixth aspect of the invention there is provided a process for creating a haptic response using a textile article according the third aspect of the invention comprising the steps of a) connecting the haptic feedback device of the textile article to a fluid source; and b) inflating the expandable material over at least one aperture with fluid from the fluid source.

### DETAILED DESCRIPTION OF THE INVENTION

In order that the invention may be more clearly understood an embodiment/embodiments thereof will now be described, by way of example only, with reference to the accompanying drawings, of which:

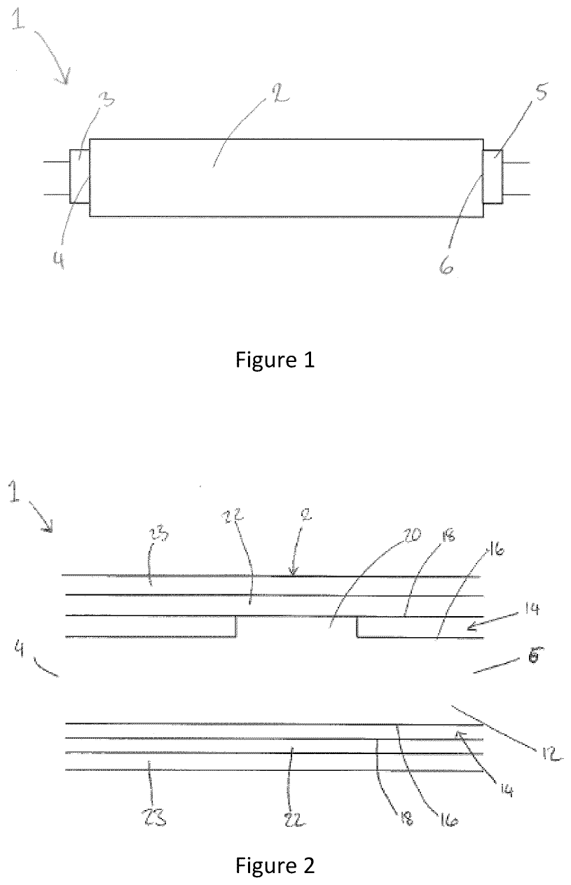

FIG. 1 illustrates a side view of a first embodiment of a haptic feedback device of the first aspect of the invention, in the form of a tube.

FIG. 2 illustrates a side cross-sectional view of part of the first embodiment of a haptic feedback device of the first aspect of the invention as shown in FIG. 1.

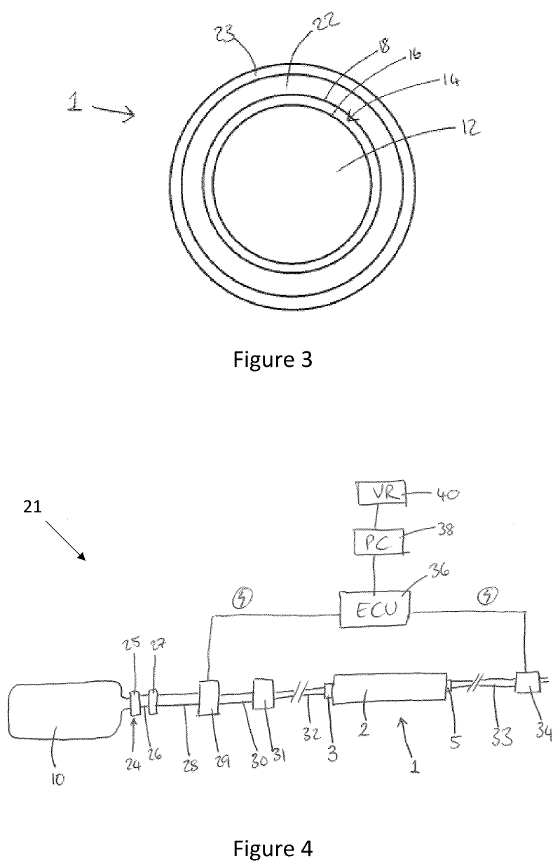

FIG. 3 illustrates an axial cross-sectional view of part of the first embodiment of the haptic feedback device of the first aspect of the invention as shown in FIG. 1.

FIG. 4 illustrates a first embodiment of a haptic feedback system of the second aspect of the invention of the invention comprising one haptic feedback device of the first aspect of the invention.

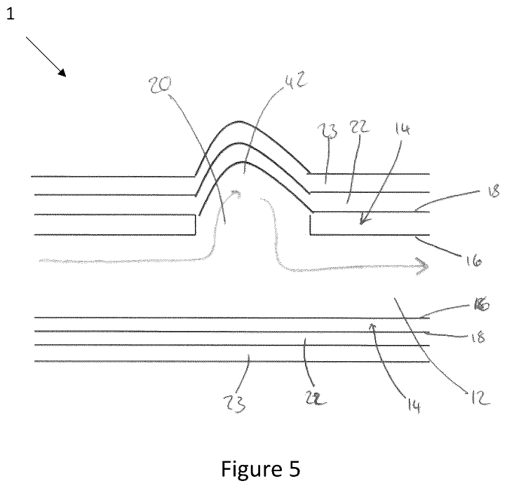

FIG. 5 illustrates a side cross-sectional view of the first embodiment of the haptic feedback device of the first aspect of the invention shown in FIGS. 1, 2 and 3, wherein the fluid, as illustrated by arrows, has entered the device and created a protrusion.

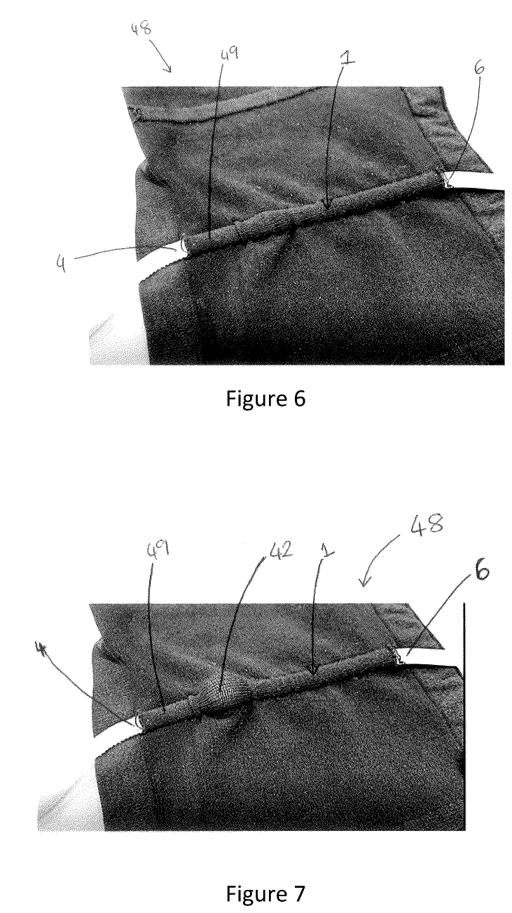

FIG. 6 illustrates a first embodiment of a textile article according to the third aspect of the invention, comprising one haptic feedback device of the first aspect of the invention inserted into a channel.

FIG. 7 illustrates the first embodiment of the textile article according to the third aspect of the invention comprising one haptic feedback device as shown in FIG. 6 wherein fluid has entered the haptic feedback device and an inflated protrusion has been created.

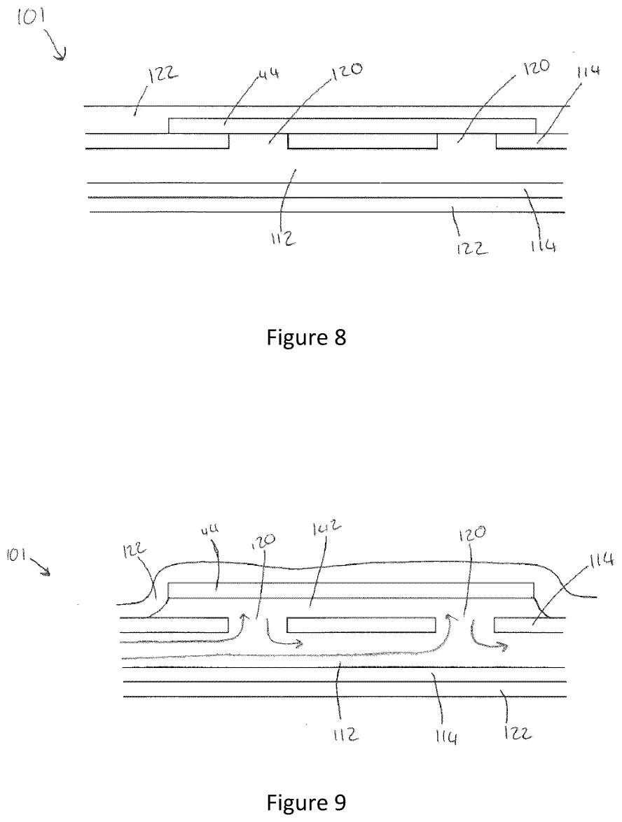

FIG. 8 illustrates a side cross-sectional view of a second embodiment of a haptic feedback device of the first aspect of the invention, in the form of a tube, wherein the haptic feedback device comprises a flexible plate located between the non-expandable material layer and the expandable material layer.

FIG. 9 illustrates a side cross-sectional view of the second embodiment of the haptic feedback device of the first aspect of the invention shown in FIG. 8, wherein the fluid, as illustrated by arrows, has entered the device and created a protrusion.

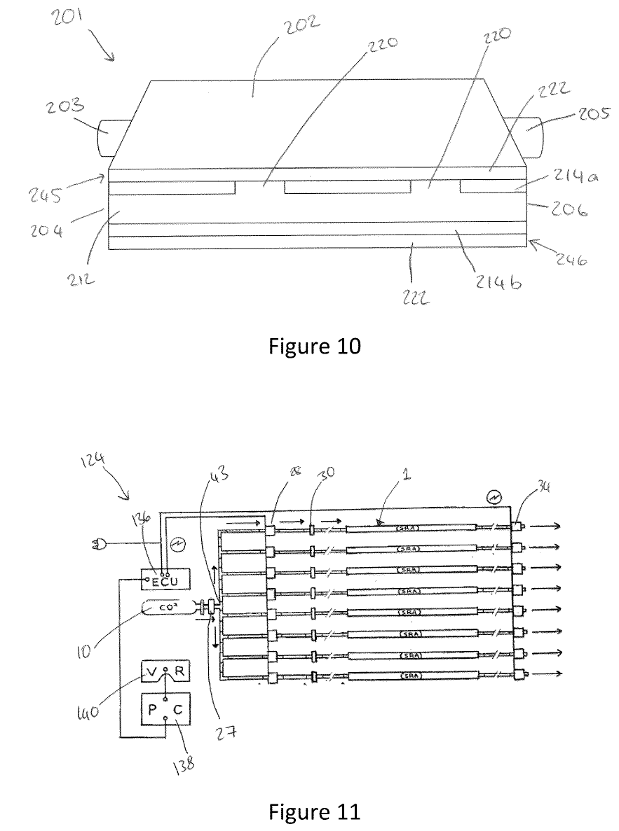

FIG. 10 illustrates a side cross-sectional view of a third embodiment of a haptic feedback device of the first aspect of the invention, in the form of a sheet.

FIG. 11 illustrates a second embodiment of a haptic feedback system of the second aspect of the invention comprising multiple haptic feedback devices.

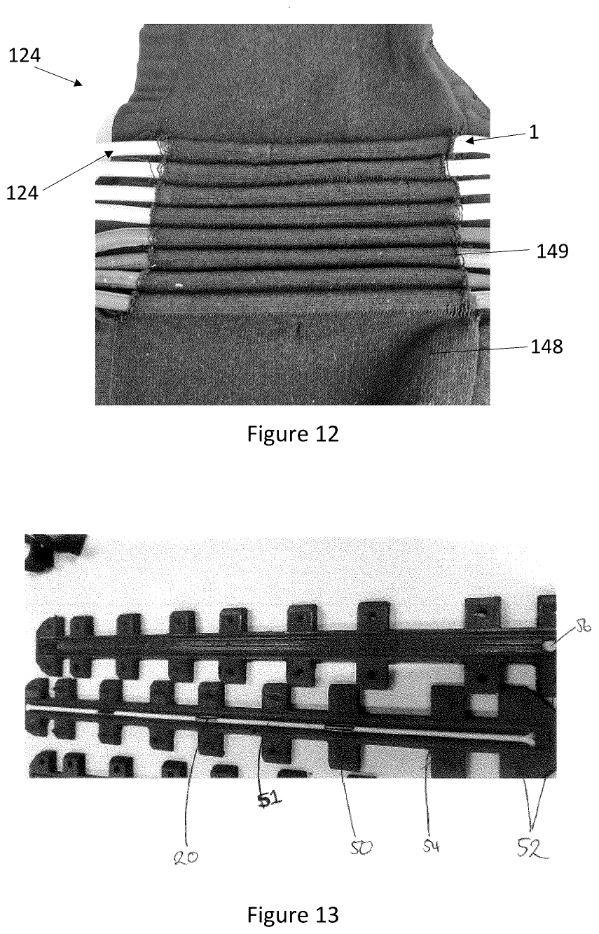

FIG. 12 illustrates a second embodiment of a textile article according to the third aspect of the invention, comprising multiple haptic feedback devices of the first aspect of the invention inserted into multiple channels.

FIG. 13 is a photograph of a two-part mould suitable for use in the manufacturing a haptic feedback device according to the invention.

A first embodiment of a haptic feedback device of the first aspect of the invention is illustrated by way of example in FIGS. 1, 2 and 3. FIG. 1 illustrates a side view of the device, FIG. 2 illustrates a side cross-sectional view of part of the device, and FIG. 3 illustrates an axial, or end cross-sectional view. The haptic feedback device (1) comprises a body (2) in the form of a tube with two ends. The haptic feedback device (1) comprises a fluid inlet connector (3) located at the upstream end of the body (4) and a fluid outlet connector (5) located at the downstream end of the body (6) The inlet connector (5) is in turn connected to a fluid source.

The tube comprises a cavity in the centre (12) which is surrounded by a layer of non-expandable material (14). The non-expandable material (14) may be polyester, nylon, zylon, cotton, Kevlar, carbon fibre, cordura, silk or any inextensible material. The non-expandable material (14) comprises an interior surface (16) and an exterior surface (18). The interior surface (16) is in fluid contact with the central cavity (12). The non-expandable material layer (14) comprises at least one aperture (20) extending through the non-expandable material from the interior (16) to the exterior surface (18). A layer of expandable material (22) is located on the exterior surface (18) of the non-expandable material (14) and covering the apertures (20) thereby creating portions of the expandable material (22) that are free of non-expandable material (14) beneath them, and which are in fluid contact with the cavity (12) via the apertures (20). The expandable material (22) may be partially embedded within the outer surface (16) of the non-expandable material (14) at the points where the expandable material (22) is located at the exterior surface (18) of the non-expandable material (14) and wherein the non-expandable material (14) is a porous material such as a textile. The expandable material may be a polymer such as polydimethylsiloxanes (PDMS), polyethylene, polyvinyl or polyurethane (PU) based elastomeric membranes or any combination thereof.

The layer of expandable material (22) is surrounded by a second layer of textile with limited expandability (23) such as spandex, Lycra® or elastane, which acts as an extensibility limiter such that it limits the extensibility of the expandable material layer (22). The textile with limited expandability (23) may be a textile which may expand as the expandable material layer (22) inflates but does not expand to the same extent as the expandable material layer (22) such that it prevents over inflation of the expandable material layer (22). Therefore, the textile with limited expandability (23) is more expandable than the non-expandable material (14) but less expandable than the expandable polymer material (22).

The haptic feedback device (1) is arranged within a haptic feedback system (23) of the second aspect of the invention as illustrated in FIG. 4. The haptic feedback system (23) comprises a fluid source in the form of a miniature compressed carbon dioxide cannister (10), but the fluid source may be any suitable source such as compressed air, a water tank or the like. An example of a suitable compressed CO₂ cannister is a Milbro ASG gas capsule 12 g or 88 g CO₂. The gas cannister (10) comprises an outlet (24) comprising an adjustable flow rate screw valve (25) attached to the gas cannister (10). The adjustable flow rate screw valve (25) is connected by way of a pipe (26) to a one-way valve (27) which maintains array pressure between actuation periods. The one-way valve (27) is then connected by way of a pipe (28) to a one-way inlet slave solenoid valve (29) for example a Solenoid Pull 12V such as an Adafruit Industries LLC 412 solenoid valve or a 6V Air Valve with 2-pin JST PH Connector. It can be understood that a two-way or three-way solenoid valve may also be used. The solenoid valve (29) is connected by way of a pipe (30) to another one-way valve (31). The one-way valve (31) is then connected by way of a pipe (32) to the fluid inlet connector (3) of the haptic feedback device (1).

The haptic feedback device body fluid outlet connector (5) is connected by way of a pipe (33) to an exhaust solenoid (34) which vents the fluid to the atmosphere.

The inlet slave solenoid valve (29) and the exhaust solenoid valve (34) are electronically connected to an electronic control unit (36) which in turn is connected to a computer (38) which is connected to a virtual reality device (40) such as a virtual reality headset.

In use the haptic feedback system (21) is arranged such that it creates a haptic feedback response according to the process of the fourth aspect of the invention. The gas cannister (10) provides fluid for the system (21). The fluid flow is controlled by the adjustable flow rate screw valve (25). Fluid will flow to the one-way solenoid valve (29) through the one-way valve (27). The one-way solenoid valve (29) is electronically controlled by the electronic control unit (36) such that the duration of the air flow, the pressure and the timing can be controlled such that the haptic feedback created within the haptic feedback device is created as desired. When the solenoid valve (29) is open, fluid will flow through the connected pipe (30), the valve (31) and the final pipe (32) before entering the haptic feedback device (1) through the inlet connector (3).

The fluid flow through the haptic feedback device (1) in use is illustrated as arrows in FIG. 5. After entering the haptic feedback device (1) the fluid will be in contact with the non-expandable material (14) surrounding the cavity (12) which is now filled with fluid. The fluid will also enter the aperture or apertures (20). When entering the aperture(s), the fluid will create pressure on the expandable material layer (22) located above each aperture (20) such that the expandable material (22) will inflate and create a protrusion (42) as illustrated in FIG. 5. In use, when the haptic feedback device (1) is in contact with the user's skin or a body part, the inflated protrusion (42) creates a haptic feedback response. The electronic control unit (36) may control the flow of the fluid such that each aperture (20) can independently inflate or deflate to create independently controlled protrusions (42) such that in use, the desired haptic response can be created where the protrusion (42) is in contact with the user. Once the haptic response has been created, the exhaust solenoid (34) may be opened as controlled by the electronic control unit (36) such that the fluid can be vented out of the system. In this embodiment wherein the fluid is carbon dioxide, the carbon dioxide may be vented out into the air. The expandable material layer (22) of the haptic feedback device (1) then results to its original, deflated arrangement such that it can be inflated again to create another haptic response.

The haptic feedback system (21) may be arranged within a textile article according to the third aspect of the invention. An embodiment of the textile article is illustrated in FIG. 6 and FIG. 7. The textile article (48) may comprise a channel (49) in which the haptic feedback device (1) is inserted. FIG. 7 illustrates the textile article in use. When fluid enters the haptic device, the protrusion (42) in the haptic feedback device (1) is inflated, and the portion of the textile article above the protrusion moves, creating a haptic response.

A second embodiment of a haptic feedback device of the invention is illustrated by way of example in FIG. 8. The haptic feedback device (101) comprises a flexible plate (44) located between a non-expandable material layer (114) and an expandable material layer (122). The flexible plate is located above two or more apertures (120). The haptic feedback system comprising the haptic feedback device (101) may be essentially the same as previous embodiments and the method of use of the haptic feedback system may be essentially the same.

The fluid flow through the haptic feedback device (1) in use is illustrated as arrows in FIG. 9. The fluid enters the apertures (120) and pushes the plate (44) outwards which in turn moves the expandable material (122) located above the plate (44) outwards. This creates a protrusion (142) that is larger than the protrusion (42) formed in the haptic feedback device of the first embodiment of the invention (1) which is illustrated in FIG. 5. The additional flexible plate (44) in the second embodiment of the invention is larger than the sum of the individual apertures (120). This may be advantageous because it allows a haptic feedback response with a larger diameter to be created which may be desired.

A third embodiment of a haptic feedback device of the invention is illustrated in FIG. 10. The haptic feedback device (201) comprises a body (202) which is a sheet. The sheet comprises a central cavity (212) with a planar top (245) and a planar bottom (246). A layer of non-expandable material (214a) comprising multiple apertures (220) is located at the top (245) of the central cavity (212). A layer of non-expandable material (214b) is located at the bottom (246) of the central cavity (212). A layer of expandable material (222) is located above the layers of non-expandable material (214a and 214b) and above the apertures (220). The haptic feedback device (201) comprises an inlet connector (203) located at the upstream end of the body (204) and an outlet connector (205) located at the downstream end of the body (206). This embodiment may be advantageous because a sheet like haptic feedback device would have a larger surface area such that a haptic feedback response can be created over a larger surface area.

A second embodiment of the haptic feedback system of the invention is illustrated in FIG. 11. The haptic feedback system (121) comprises a plurality of haptic feedback devices (1). The inlet connector (3) of each haptic feedback device (1) may be connected to a one-way valve (30) and then to a solenoid valve (28) such that there is one one-way valve (30) and one solenoid valve (28) for each haptic feedback device. The multiple solenoid valves (28) are connected to each other at a single point (43) which is then connected through a one-way valve (27) to a gas cannister (10). The gas cannister (10) is a miniature compressed carbon dioxide cannister. This arrangement is advantageous because the plurality of haptic feedback devices (1) would mean a haptic response can be created over a larger surface area. The solenoid valves (28) are connected to an electronic control unit (136) which in turn is connected to a computer (138). The computer (138) transmits and receives signals to electronically actuate the valves (28, 34) via the electronic control unit (136). The electronically actuated valves (28, 34) may be controlled independently such that the inflation of each haptic feedback device (1) may be independent of the others which creates a more controlled and desired haptic response.

The haptic feedback system (124) according to the second embodiment of the invention may be attached to a textile article (148) as illustrated in FIG. 12. The haptic feedback devices (1) of the haptic feedback system (124) are inserted into multiple channels (149) located inside or outside of the textile article (148). The textile article (148) may be a wearable item such as a glove, a shirt, a sleeve or any other fabric wearable item.

The method of use of the second embodiment of the invention is essentially the same as the first embodiment of the invention with the exception that the haptic feedback response of each of the haptic feedback devices (1) can be individually controlled by independent control of each of the solenoid valves (28, 34) which are connected to the electronic control unit (136) which is connected to a computer (138) and optionally a virtual reality headset (140). This embodiment may be advantageous because multiple haptic feedback devices (1) could be positioned at the desired location of the textile article (148) and individually controlled such that a precisely located haptic response can be created within a larger surface area.

A third embodiment of a haptic feedback system of the invention comprises a miniature air pump instead of a gas cannister. An example of a suitable miniature air pump is a 4.5V and 1.8 LPM Air Pump and Vacuum DC Motor (ZR320-02PM). The other aspects of the embodiment are essentially the same as the earlier embodiments and the method of use is essentially the same.

A second embodiment of a textile article of the invention comprises a tubular haptic feedback device connected within a haptic feedback system wherein the haptic feedback system is according to any previous embodiment. The haptic feedback device is integrated into the textile article by means of knitting or weaving. The method of use is essentially the same as previous embodiments. This embodiment is advantageous because it does not require additional channels, pockets or pouches in the textile article. It is also advantageous because the haptic feedback device may be located at any point in the textile article such that the haptic feedback response can be accurately located at any point in the textile article.

### METHODS OF MANUFACTURING HAPTIC FEEDBACK DEVICES AND SYSTEMS OF THE INVENTION

Haptic feedback devices of the invention may be manufactured by the following method. It should be understood that the method of manufacturing as disclosed below are by way of example only.

### Example 1: Degassing Bath and Then Injection

The non-expandable material layer of the haptic feedback devices of the invention may be manufactured as follows and as supported by FIG. 13. A non-expandable material (14) in the form of a non-expandable textile tube (51) was made using a braided, knitted, double covered, non-woven, woven, or embroidered process. A removable solid core (50) was inserted into the non-expandable textile tube (51) component. Optionally, feeder rollers may be used to assist with this. The internal dimensions of the non-expandable textile tube (51) should relatively match the outer dimensions of the solid core. A laser, a blade or purpose made die was used to selectively remove regions of non-expandable textile tube (51) such that apertures (20) are created. The shape and dimensions of the apertures (20) will depend on the application and modes of use.

The solid core (50) with the non-expandable textile tube (51) was degassed in a vacuum chamber to remove entrapped air from the textile/core components. The degassed removable solid core (50) with the non-expandable textile tube (51) was placed inside a two-part mould (52) as illustrated in FIG. 13. The two-part mould (52) was centrally secured at each end with a positioning/locator cradle.

An expandable material was then provided. The expandable material used was an elastomeric polymer. The elastomeric polymer was degassed and then injected in the mould cavity (54). Elastomeric polymer was injected into the mould cavity (54) under pressure until the excess material evacuated at the other end of the mould cavity (54), to remove entrapped air. The elastomeric polymer entered the degassed pores of the porous non-expandable textile tube (51) such that it was partially embedded or encapsulated within the non-expandable textile tube (51). This created a composite elastomeric polymer-soaked textile mounted on the removable solid core. The removable core prevented the polymer from substantially entering the tube through the apertures (20) thereby resulting in a layer of expandable material located on the non-expandable textile tube (51) and within and above the apertures (20).

Then the assembly was left to cure. Once cured the assembly was removed from the mould (52) and the solid core (50) was withdrawn to create the hollow tube.

### Example 2: Additional Expandable Material Layer

An additional step beyond example 1 may comprise adding a second expandable textile layer on the exterior surface of the expandable polymer layer. The expandable textile layer or tube may be manufactured separately to the expandable material layer and the structure comprising the expandable material layer may be inserted into the expandable textile layer or tube.

The above embodiments are described by way of example only. Many variations are possible without departing from the scope of the invention as defined in the appended claims.

1. A haptic feedback device comprising a body having a body cavity surrounded by a layer of non-expandable material having an interior and exterior surface and which comprises at least one aperture therethrough, and an expandable material located on the exterior surface or interior surface of the non-expandable material and covering at least one aperture, and wherein the device further comprises at least one connector for connecting the device, in use, to a source of fluid.

2. The haptic feedback device according to claim 1 wherein the expandable material comprises a layer which surrounds the non-expandable material.

3. The haptic feedback device according to claim 1 wherein the expandable material is located on the exterior surface of the non-expandable material.

4. The haptic feedback device according to claim 1 wherein the non-expandable material is a textile material.

5. The haptic feedback device according to claim 1 wherein the expandable material is an elastomeric material.

6. The haptic feedback device according to claim 1 wherein the haptic feedback device is a tube.

7. The haptic feedback device according to claim 1 wherein the haptic feedback device is essentially a sheet.

8. The haptic feedback device according to claim 1 wherein the haptic feedback device comprises a second expandable material located above the expandable material wherein the second expandable material is less expandable than the expandable material.

9. A haptic feedback system comprising the haptic feedback device according to claim 1 connected to a fluid source.

10. The haptic feedback system according to claim 9 wherein the fluid is a gas.

11. The haptic feedback system according to claim 9 wherein the fluid is a compressed gas, for example compressed air or compressed carbon dioxide.

12. (canceled)

13. The haptic feedback system according to claim 9 wherein the system comprises at least two haptic feedback devices.

14. The haptic feedback system according to claim 9 wherein the system further comprises an electronic control unit which, in use, is arranged to send electronic signals that actuate expansion and contraction of the expandable material.

15. A textile article comprising the haptic feedback device of claim 1.

16. The textile article according to claim 15 wherein the textile article is further connected to a fluid source.

17. (canceled)

18. The textile article according to claim 15 wherein the textile article comprises a channel or pocket in which is located the haptic feedback device

19. The textile article according to claim 15 wherein the haptic feedback device is braided, woven or knitted into the textile article.

20. A process for creating a haptic response using the haptic feedback device according to claim 1 comprising the steps of a) connecting the haptic feedback device to a fluid source; and b) inflating the expandable material over at least one aperture with fluid from the fluid source.

21. (canceled)

22. (canceled)

23. The process for creating a haptic response according to claim 20 comprising multiple haptic feedback devices and wherein the expandable material of each haptic feedback device is inflated independently from the other haptic feedback devices.

24. The process according to claim 20 wherein step b) comprises providing electronic signals to actuate the, or each, haptic feedback devices to inflate or deflate the, or each, haptic feedback device.
